(19) United States
(12) Patent Application Publication
(10) Pub. No.: US 2019/0154661 A1
(43) Pub. Date: May 23, 2019

(54) METHODS OF IDENTIFYING CFTR MODULATORS

(71) Applicant: Proteostasis Therapeutics, Inc., Boston, MA (US)

(72) Inventors: John Miller, Worcester, MA (US); Lawrence J. Drew, II, North Grafton, MA (US); Po-Shun Lee, Brookline, MA (US)

(21) Appl. No.: 16/300,219

(22) PCT Filed: May 9, 2017
(86) PCT No.: PCT/US2017/031754

§ 371 Date: Nov. 9, 2018

Related U.S. Application Data
(60) Provisional application No. 62/333,537, filed on May 9, 2016.

(51) Int. Cl. G01N 33/50; G01N 33/52

(52) U.S. Cl. CPC G01N 33/502; G01N 33/52; G01N 2800/382; G01N 2500/10; G01N 2500/02

(57) ABSTRACT

The present disclosure is directed to methods of identifying CFTR modulators using non-mutant or mutant CFTR expressing cells in the presence of a CFTR amplifier compound.

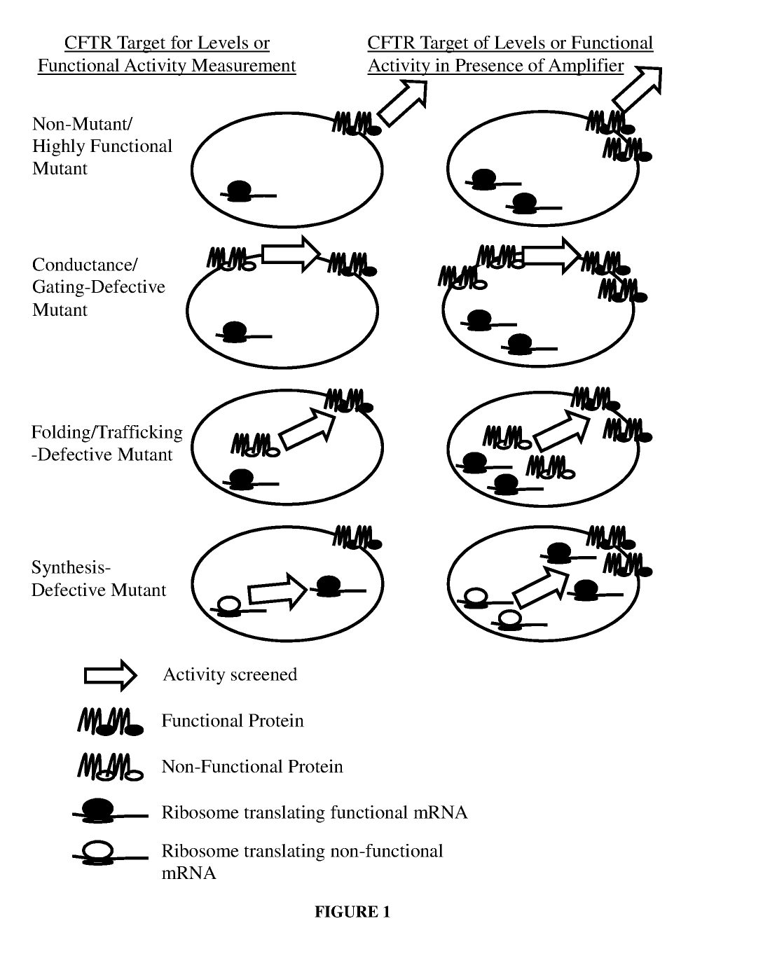

### CROSS REFERENCE TO RELATED APPLICATIONS

This application is a national stage filing under 35 U.S.C. § 371 of PCT/US2017/031754, filed on May 9, 2017, which claims the benefit of, and priority to, U.S. provisional application number 62/333,537, filed on May 9, 2016; the content of which is hereby incorporated by reference herein in its entirety.

### BACKGROUND

Cystic Fibrosis (CF) is caused by mutations in the cystic fibrosis transmembrane conductance regulator (CFTR) gene which encodes a multi-membrane spanning epithelial chloride channel (Riordan et al., Annu Rev Biochem 77, 701-26 (2008)). Approximately ninety percent of patients have a deletion of phenylalanine (Phe) 508 (ΔF508) on at least one allele. This mutation results in disruption of the energetics of the protein fold leading to degradation of CFTR in the endoplasmic reticulum (ER). The ΔF508 mutation is thus associated with defective folding and trafficking, as well as enhanced degradation of the mutant CFTR protein (Qu et al., J Biol Chem 272, 15739-44 (1997)). The loss of a functional CFTR channel at the plasma membrane disrupts ionic homeostasis (Cl⁻, Na⁺, HCO₃⁻) and airway surface hydration leading to reduced lung function. Reduced periciliary liquid volume and increased mucus viscosity impede mucociliary clearance resulting in chronic infection and inflammation, phenotypic hallmarks of CF disease (Boucher, J Intern Med 261, 5-16 (2007)). In addition to respiratory dysfunction, ΔF508 CFTR also impacts the normal function of additional organs (pancreas, intestine, gall bladder), suggesting that the loss-of-function impacts multiple downstream pathways that will require correction.

In addition to cystic fibrosis, mutations in the CFTR gene and/or the activity of the CFTR channel has also been implicated in other conditions, including for example, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), dry eye disease, Sjogren's syndrome and chronic sinusitis, cholestatic liver disease (e.g. Primary biliary cirrhosis (PBC) and primary sclerosing cholangitis (PSC)) (Sloane et al. (2012), PLoS ONE 7(6): e39809.doi:10.1371/journal.pone.0039809; Bombieri et al. (2011), J Cyst Fibros. 2011 June; 10 Suppl 2:S86-102; (Albert et al. (2008), Clinical Respiratory Medicine, Third Ed., Mosby Inc.; Levin et al. (2005), Invest Ophthalmol Vis Sci., 46(4):1428-34; Froussard (2007), Pancreas 35(1): 94-5), Son et al. (2017) J Med Chem 60(6):2401-10).

There remains a need in the art for methods of identifying CFTR modulator compounds that may be useful for treating CF, other CFTR-related diseases, and other maladies of protein misfolding.

### SUMMARY

This disclosure is directed, at least in part, to a method of identifying a CFTR modulator compound using a CFTR amplifier to increase the levels of CFTR so that the CFTR modulator compound has more substrate to act upon, comprising:

incubating a set of test compounds and a CFTR amplifier compound with cells expressing non-mutant or mutant CFTR; and

measuring non-mutant or mutant CFTR levels and/or activity in response to the test compounds in the presence of the amplifier.

### BRIEF DESCRIPTION OF THE DRAWINGS

The Figure depicts a method of identifying a CFTR modulator compound using an amplifier compound. The left panels show the activity derived from a modulator being sought in the screen. The right panels show that the amplifier provides more substrate CFTR species for each of the modulators to act upon.

### DETAILED DESCRIPTION

As used herein, the words “a” and “an” are meant to include one or more unless otherwise specified. For example, the term “an agent” encompasses both a single agent and a combination of two or more agents.

The term “modulating” encompasses increasing, enhancing, inhibiting, decreasing, suppressing, and the like. The terms “increasing” and “enhancing” mean to cause a net gain by either direct or indirect means. As used herein, the terms “inhibiting” and “decreasing” encompass causing a net decrease by either direct or indirect means.

Disclosed herein is a method of identifying a CFTR modulator compound, comprising one or more of the following:

incubating a set of test compounds in combination with a CFTR amplifier compound with cells expressing a non-mutant or mutant CFTR in incubation media;

measuring CFTR levels and/or activity; and

identifying one or more compounds from the set of test compounds that modulate CFTR levels or activity, thereby identifying CFTR modulator compounds.

For example, disclosed herein is a method of identifying a CFTR modulator compound, comprising one or more of the following:

incubating a set of test compounds in the presence of a CFTR amplifier compound with a cell line expressing non-mutant or mutant CFTR in incubation media;

measuring CFTR levels and or activity in response to the test compounds; and

identifying a modulator of CFTR activity based on the ability to act on the levels and or activity of CFTR in the presence of the CFTR amplifier compound.

In some embodiments, disclosed cells may express non-mutant or at least one mutant CFTR, selected for example from the group consisting of ΔF508, S549N, G542X, G551D, R117H, N1303K, W1282X, R553X, 621+1G>T, 1717-1G>A, 3849+10kbC>T, 2789+5G>A, 3120+1G>A, I507del, R1162X, 1898+1G>A, 3659delC, G85E, D1152H, R560T, R347P, 2184insA, A455E, R334W, Q493X, Y122X, K710X, R553X, R709X, R1158X and R1162X and 2184delA. Contemplated CFTR mutation(s) may be from one or more classes, such as without limitation, Class I CFTR mutations, Class II CFTR mutations, Class III CFTR mutations, Class IV CFTR mutations, Class V CFTR mutations, and Class VI mutations. Contemplated cell line CFTR genotypes may include, without limitation, homozygote mutations (e.g., ΔF508/ΔF508 and R117H/R117H) and compound heterozygote mutations (e.g., ΔF508/G551D; ΔF508/A455E; AF508/G542X; Δ508F/W1204X; R553X/W1316X; W1282X/N1303K, 591Δ18/E831X, F508del/R117H/N1303K/3849+10kbC>T; Δ303K/384; and DF508/G178R).

In certain embodiments, the mutation is a Class I mutation, e.g., a G542X; a Class II/I mutation, e.g., a ΔF508/G542X compound heterozygous mutation. In other embodiments, the mutation is a Class III mutation, e.g., a G551D; a Class II/Class III mutation, e.g., a ΔF508/G551D compound heterozygous mutation. In still other embodiments, the mutation is a Class V mutation, e.g., a A455E; or a Class II/Class V mutation, e.g., a ΔF508/A455E compound heterozygous mutation.

In some embodiments, a contemplated cell's CFTR genotype may include, without limitation, one or more Class I CFTR mutations, one or more Class II CFTR mutations, one or more Class III CFTR mutations, one or more Class IV CFTR mutations, one or more Class V CFTR mutations, or one or more Class VI CFTR mutations. In certain embodiments, a contemplated cell's CFTR genotype may include, without limitation, one or more homozygote mutations (e.g., ΔF508/ΔF508 or R117H/R117H) and/or one or more compound heterozygote mutations (e.g., ΔF508/G551D; ΔF508/A455E; AF508/G542X; Δ508F/W1204X; R553X/W1316X; W1282X/N1303K; F508del/R117H; N1303K/3849+10kbC>T; ΔF508/R334W; DF508/G178R, and 591Δ18/E831X). In certain embodiments, a cell's CFTR genotype includes a Class I mutation, e.g., a G542X Class I mutation, e.g., a ΔF508/G542X compound heterozygous mutation. In other embodiments, a cell's CFTR genotype includes a Class III mutation, e.g., a G551D Class III mutation, e.g., a ΔF508/G551D compound heterozygous mutation. In still other embodiments, a cell's CFTR genotype includes a Class V mutation, e.g., a A455E Class V mutation, e.g., a ΔF508/A455E compound heterozygous mutation.

In certain embodiments, incubating may occur for about 24 hours. In other embodiments, incubating may occur from about 48 hours to about 72 hours.

In some embodiments, a method disclosed herein may comprise measuring CFTR activity. In some embodiments, disclosed cells may e.g., exogenously express a detectable marker such as a fluorescent protein (e.g. halide-sensitive yellow fluorescent protein (hsYFP)). Other detectable markers may include isotopic labels, optically detectable dyes and/or markers (e.g. fluorophores, and the like). Contemplated fluorescent proteins include, but are not limited to, TagBFP, mTagBFP2, Azurite, EBFP, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, Monomeric Azami Green, TagGFP2, mUKG, mWasabi, Clover, mNeonGreen, EYFP, Citrine, Venus, SYFP2, TagYFP, Monomeric Kusabira-Orange, mKOk, mKO2, mOrange, mOrange2, PSmOrange, mRaspberry, mCherry, mStrawberry, mTangerine, tdTomato, TagRFP, TagRFP-T, mApple, mRuby, and mRuby2, mPlum, HcRed-Tandem, mKate2, mNeptune, NirFP, TagRFP657, mKeima Red, LSS-mKatel, LSS-mKate2, mBeRFP, PA-GFP, PAmCherryl, PATagRFP, Kaede, KikGR1, PS-CFP2, mEos2, and mEos3.2. Fluorophores may include, but are not limited to, one or more of fluorescein, rhodamine, Oregon green, eosin, Texas red, coumarin, hydroxycoumarin, aminocoumarin, methoxycoumarin, cyanine, indocarbocyanine, oxacarbocyanine, thiacarbocyanine, merocyanine, Seta, SeTau, dansyl and prodan derivatives, pyridyloxazole, nitrobenzoxadiazole, benzoxadiazole, anthraquinones (including DRAQ5, DRAQ7 and CyTRAK Orange), cascade blue, Pacific blue, Pacific orange, Lucifer yellow, R-phycoerythrin (PE), PE-Cy5 conjugates, PE-Cy7 conjugates, Red 613, PerCP, TruRed, FluorX, BODIPY-FL, Cy2, Cy3, Cy3B, Cy3.5, Cy5, Cy5.5, Cy7, TRITC, X-rhodamine, lissamine rhodamine B, allophycocyanin (APC), APC-Cy7 conjugates, Nile red, Nile blue, cresyl violet, oxazine 170, proflavin, acridine orange, acridine yellow, auramine, crystal violet, malachite green, porphin, phthalocyanine, and bilirubin.

In some embodiments, measuring CFTR activity may comprise measuring the fluorescence of a hsYFP. In a further embodiment, measuring CFTR activity may further comprise adding a salt such as a halide salt (e.g. lithium chloride, lithium bromide, lithium iodide, sodium chloride, sodium bromide, sodium iodide, potassium chloride, potassium bromide, potassium iodide) to the cells, and detecting a signal associated with that, for example, detecting hsYFP signal quenching, thereby ascertaining the rate at which the halide salt is transported into the cells and CFTR activity.

In certain embodiments, measuring CFTR activity may comprise measuring the chloride transport in electrophysiological assays.

In various embodiments, disclosed herein is a method of identifying readthrough modulators of CFTR premature termination codon (PTC) mutations, e.g., CFTR production correctors, as evidenced by the trafficking and cell surface expression of full-length CFTR. Without being bound by theory, it is believed that CFTR production correctors may instruct ribosomes to read-through premature termination codons (PTCs) during mRNA translation, thereby ensuring that a full-length functional CFTR protein is translated, produced, and transported to the Golgi for further processing.

In various embodiments, a method disclosed herein may comprise measuring CFTR levels. In certain embodiments, measuring, e.g. measuring CFTR levels, may comprise measuring CFTR protein levels, for example, full-length CFTR protein levels, within the cell (e.g., internally) or at the cell surface or, for example, at the cell membrane.

In some embodiments, disclosed cells may exogenously express, for example, a detectable CFTR fusion protein having at least one CFTR mutation, e.g., a CFTR premature termination codon (PTC) mutation. Examples of such PTC mutations may include, but are not limited to, e.g. G542X, W1282X, Y122X, K710X, R553X, R709X, R1158X and R1162X.

In certain embodiments, measuring CFTR levels, e.g., measuring CFTR levels within the cell or at the cell surface, may comprise, for example, detecting the luminescence of a disclosed CFTR fusion protein using, e.g., a luminescence assay. Examples of such CFTR fusion proteins may include, but are not limited to, e.g. CFTR-HRP (CFTR-horseradish peroxidase) and, e.g., CFTR-luciferase, e.g., CFTR-firefly luciferase.

In certain embodiments, measuring CFTR levels, e.g., measuring CFTR levels within the cell or at the cell surface, may comprise, for example, detecting the fluorescence of a disclosed CFTR fusion protein using, e.g.,, a fluorescence assay. Examples of such CFTR fusion proteins may include, but are not limited to, e.g. CFTR-RFP (CFTR-red fluorescence protein), CFTR-YFP (CFTR-yellow fluorescence protein) and, e.g., CFTR-GFP (CFTR-green fluorescence protein).

In some embodiments, disclosed cells may exogenously express, for example, a detectable, epitope-tagged CFTR protein having at least one CFTR mutation, e.g., a CFTR premature termination codon (PTC) mutation. Examples of such PTC mutations may include, but are not limited to, e.g. G542X, W1282X, Y122X, K710X, R553X, R709X, R1158X and R1162X.

In certain embodiments, measuring CFTR levels, e.g, measuring CFTR levels within the cell or at the cell surface, may comprise, for example, detecting a diclosed epitope-tagged CFTR protein using, e.g., an enzyme-linked immunosorbent assay (ELISA). Examples of such epitope-tagged CFTR proteins may include, but are not limited to, e.g. CFTR-HA (CFTR-hemagglutinin) and, e.g., CFTR-FLAG.

In some embodiments, measuring CFTR levels within the cell or at the cell surface may comprise measuring the levels of CFTR protein using e.g., western blotting or enzyme-linked immunosorbent assay. In certain embodiments, measuring CFTR levels within the cell or at the cell surface may comprise measuring the levels of CFTR mRNA using e.g., quantitative reverse transcriptase polymerase chain reaction or similar method.

In some embodiments, a disclosed set of test compounds may further include a reference CFTR production corrector compound as a positive control compound. In some embodiments, an incubating step in a method disclosed herein may further comprise incubating a reference CFTR production corrector in combination with a CFTR amplifier compound with cells expressing a non-mutant or mutant CFTR in incubation media, thereby serving as a positive control. Examples of reference CFTR production correctors may include, but are not limited to, e.g., ataluren, NB124, and G418 (geneticin). For example, a contemplated reference CFTR production corrector may be, e.g, G418 (geneticin).

In certain embodiments, an incubating step in a method disclosed herein may further comprise adding a CFTR potentiator compound to the cells. In some embodiments, an incubating step in a method disclosed herein may further comprise incubating a set of test compounds in combination with a CFTR potentiator compound and a CFTR amplifier compound with cells expressing a non-mutant or mutant CFTR in incubation media. Non-limiting examples of CFTR potentiators include VX-770 (ivacaftor), deuterated ivacaftor, genistein, GLPG1837/ABBV-974, GLPG2451, and QBW251. For example, a contemplated CFTR potentiator may be selected from the group consisting of ivacaftor, genistein, QBW251, GLPG2451, and GLPG1837. In certain embodiments, adding the CFTR potentiator may further comprise removing the incubation media, adding forskolin, and further incubating, e.g., for about 1 hour.

In certain embodiments, disclosed methods of identifying a CFTR modulator may include indentifying a CFTR modulator that may have a different mechanism of action than the CFTR amplifier used as part of the detection method. For example, provided herein are methods of identifying a CFTR modulator wherein the CFTR modulator may be a CFTR potentiator, a CFTR activator, a CFTR stabilizer, and/or, for example, a CFTR corrector.

In another embodiment, disclosed herein is a method of identifying a CFTR modulator compound, comprising one or more of the following:

providing a first set and a second set of one or more test compounds, each set having the same test compounds;

incubating the first set of test compounds with a first cell line overexpressing a CFTR mutation in incubation media;

incubating the second set of test compounds with a CFTR amplifier compound and a second cell line overexpressing a CFTR mutation in incubation media, wherein the first cell line and the second cell line are the same;

optionally adding a CFTR potentiator to further activate the CFTR mutation to the first and second set of test compounds;

measuring CFTR levels and/or activity of the first and second set of test compounds; and

identifying one or more compounds in the second set of test compounds having about 5%, about 10%, about 15%, about 25%, or about 30% or more CFTR levels and/or activity when compared to the same compound in the first set of test compounds, thereby identifying the CFTR modulator compound.

### Exemplification

While this disclosure has been particularly shown and described with references to embodiments thereof, it will be understood by those skilled in the art that various changes in form and details may be made therein without departing from the scope of the disclosure encompassed by the appended claims. The disclosure is illustrated by the following examples which are not meant to be limiting in any way.

### EXAMPLE 1

Cystic fibrosis bronchial epithelial (CFBE) cells overexpressing ΔF508-CFTR and exogenously expressing halide-sensitive yellow fluorescent protein (hsYFP) were seeded into 384-well plates and incubated for 48 hours prior to compound treatment. In the first iteration the compounds of the library and the amplifier compound were added to the cells sequentially or simultaneously and incubated for a further 24 hours.

Following the 24 hour incubation with compounds, the media can be removed from the cells and the cells can be washed with PBS. Cells can be incubated for one hour in the presence of forskolin and potentiator in order to maximize activation of ΔF508-CFTR.

The fluorescence of the hsYFP was then kinetically measured in a fluorescent plate reader that monitored all 384-wells simultaneously. Ten seconds into the readout of fluorescence, sodium iodide was added to the cells, and the rate at which the sodium iodide was transported into the cells was a reflection of the ΔF508-CFTR activity. This rate was observed by hsYFP signal quenching in response to the intracellular sodium iodide.

In the assay, a hit is called for a test compound that shows greater than 25% of the positive control compound which is present in specified wells of each library plate, and is treated identically to the library compound wells also present on the plate. Examples of test compounds representing a hit are 6,8-dimethyl-2-(3-methyl-1-benzofuran-2-yl)quinoline-4-carboxylic acid, 2-(1-benzofuran-2-yl)-5-(benzyloxy)-8-methylquinoline-4-carboxylic acid, 6-chloro-8-methyl-2-(3-methylbenzofuran-2-yl)quinoline-4-carboxylic acid, 8-methyl-2-(3-methyl-1-benzofuran-2-yl)-6-(trifluoromethyl)quinoline-4-carboxylic acid, 6-fluoro-8-methyl-2-(3-methylbenzofuran-2-yl)quinoline-4-carboxylic acid, 6-bromo-8-methyl-2-(3-methyl-1-benzofuran-2-yl)quinoline-4-carboxylic acid, 6-methoxy-8-methyl-2-(3-methyl-1-benzofuran-2-yl)quinoline-4-carboxylic acid, 6-(benzyloxy)-8-methyl-2-(3-methyl-1-benzofuran-2-yl)quinoline-4-carboxylic acid, 6-ethyl-8-methyl-2-(3-methyl-1-benzofuran-2-yl)quinoline-4-carboxylic acid, and 5,8-dimethyl-2-(3-methyl-1-benzofuran-2-yl)quinoline-4-carboxylic acid.

### EXAMPLE 2

Overview

The level of CFTR at the cell surface was measured in Fisher Rat Thyroid (FRT) cells to determine the effect of amplifier on readthrough agent. FRT cells were seeded into a 384-well plate and incubated overnight prior to compound treatment. The compounds of the library or G418 (control) and the amplifier compound were added to the cells sequentially or simultaneously and incubated for a further 48 hours. Following the 48-hour incubation with compounds, the media was removed from the cells and the cells washed with PBS. ELISA substrate was added to each well and the cells were further incubated for 10 minutes. The level of CFTR was measured enzymatically using a horseradish peroxidase-sensitive luminescence assay. In the assay, a hit is called for a test compound that shows greater than 25% of the positive control compound which is present in specified wells of each library plate, and is treated identically to the library compound wells also present on the plate.

Protocol

FRT cells were harvested for seeding as follows. Cell media was aspirated from a T150 flask and the cells were washed with 10 mL PBS. To the cells was added 7 mL of 0.25% Trypsin. The cells were coated evenly. The cells were incubated at 37° C. for 10 minutes. The cells were observed under a microscope and the flask tapped gently to dislodge the cells. Typically, 95% of cells were detached after 10 minutes of incubation. Cells were incubated at 37° C. for an additional 5 minutes if cells were still attached after tapping. Trypsin was quenched with 20 mL of complete media and the contents of the flask were transferred to a 50 mL conical tube. The flask was washed with 10 mL of complete media and the solution transferred to the 50 mL conical tube containing the cell suspension. The cells were spun to a pellet at 1000 RPM for 5 minutes at room temperature. The supernatant was aspirated without disturbing the pellet and resuspended in 10 mL of complete media. A volume of 0.5 mL of cell suspension was added to a Vi-CELL cup for cell counting; typical yields are 20×10⁶ cells total at 95% confluency. Cells were seeded into a 384-well white opaque plate at 15,000 cells/well for a total volume of 20 μL using a Multidrop combi. The plates were incubated at 37° C. overnight.

Compound dosing and horseradish peroxidase (HRP) assay were performed as follows. The incubation media was warmed to 37° C. in a water bath. All compound treatments are made up at 2× concentration in complete media as listed below.

G418—stock: 50 mg/mL; final conc: 250 μg/mL; 2× conc: 500 μg/mL

Amplifier—stock: 100 mM; final conc: 10 μM; 2× conc: 20.2 μM

Vehicle—combination of water and DMSO (final DMSO conc: 0.04%)

A volume of 20 μL of incubation media containing compound was added to each well for a final volume of 40 μL using a Biomek Fx liquid handler. The plates were incubated at 37° C. for 48 hours. The plates were washed 3× with PBS (containing Ca²⁺ and Mg²⁺) on a Biotek plate washer. The PBS was aspirated to 6 μL in a plate aspirator and residual PBS was removed. A working solution of West Femto ELISA substrate was prepared by mixing equal parts of Stable Peroxide solution and Luminol/Enhancer solution. A volume of 20 μL of the working solution was added to each well using a Biomek Fx liquid handler. The plates were incubated at room temperature for 10 minutes and read on a PerkinElmer Envision plate reader using a luminescence protocol (0.2 second read/well).

### EXAMPLE 3

Ussing Measurements

Ussing measurements are used to measure CFTR activity. In this method, primary lung epithelial cells (hBEs) with a Cystic Fibrosis-causing mutation are differentiated for a minimum of 4 weeks in an air-liquid interface on SnapWell™ filter plates prior to the Ussing measurements. Cells are apically mucus-washed for 30 minutes prior to treatment with compounds. The basolateral media are removed and replaced with media containing the compound of interest diluted to its final concentration from DMSO stocks. Treated cells are incubated at 37° C. and 5% CO₂ for 24 hours. At the end of the treatment period, the cells on filters are transferred to the Ussing chamber and equilibrated for 30 minutes. The short-circuit current is measured in voltage clamp-mode (V_{hold}=0 mV), and the entire assay is conducted at a temperature of 36° C.-36.5° C. Once the voltages stabilized, the chambers are clamped, and data is recorded by pulse readings every 5 seconds. Following baseline current stabilization, the following additions are applied and the changes in current and resistance of the cells is monitored:

1. Benzamil to the apical chamber to inhibit ENaC sodium channel

2. Forskolin to both chambers to activate ΔF508-CFTR by phosphorylation.

3. VX-770 or Genistiein to the apical chamber to potentiate ΔF508-CFTR channel opening.

4. CFTRinh-172 to the apical chamber to inhibit ΔF508-CFTR Cl-conductance.

The forskolin-sensitive current and inhibitable current (that potentiated current that is blocked by CFTRinh-172) are measured as the specific activity of the ΔF508-CFTR channel, and increases in response to compound in this activity over that observed in vehicle-treated samples are identified as the correction of ΔF508-CFTR function imparted by the compound tested.

hBE Equivalent Current (Ieq) Assay

Primary lung epithelial cells with Cystic Fibrosis-causing mutations are differentiated for a minimum of 4 weeks in an air-liquid interface on Costar 24 well HTS filter plates prior to the equivalent current (Ieq) measurements. Cells are apically mucus-washed for 30 minutes prior to treatment with compounds. The basolateral media is removed and replaced with media containing the compound of interest diluted to its final concentration from DMSO stocks. Treated cells are incubated at 37° C. and 5% CO₂ for 24 hours. At the end of the treatment period, the media is changed to the Ieq experimental solution for 30 minutes before the experiment and plates are maintained in a CO₂-free incubator during this period. The plates containing the cells are then placed in pre-warmed heating blocks at 36° C.±0.5° C. for 15 minutes before measurements are taken. The transepithelial voltage (V_{T}) and conductance (G_{T}) is measured using a custom 24 channel current clamp (TECC-24) with 24 well electrode manifold. The Ieq assay measurements are made following additions with standardized time periods:

1. The baseline V_{T} and G_{T} values are measured for approximately 20 minutes.

2. Benzamil is added to block ENaC for 15 minutes.

3. Forskolin plus VX-770 (ivacaftor) are added to maximally activate ΔF508-CFTR for 27 minutes.

4. Bumetanide is added to inhibit the NaK₂Cl cotransporter and shut-off secretion of chloride.

The activity data captured is the area under the curve (AUC) for the traces of the equivalent chloride current. The AUC is collected from the time of the forskolin/VX-770 addition until the inhibition by bumetanide addition. Correction in response to compound treatment is scored as the increase in the AUC for compound-treated samples over that of vehicle-treated samples.

### INCORPORATION BY REFERENCE

All publications and patents mentioned herein, including those items listed below, are hereby incorporated by reference in their entirety for all purposes as if each individual publication or patent was specifically and individually incorporated by reference: WO/2014/210159, WO/2015/138909, WO/2015/138934, WO/2015/196071, and WO/2017/062581. In case of conflict, the present application, including any definitions herein, will control.

### Equivalents

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A method of identifying a CFTR modulator compound, comprising:
incubating a set of test compounds in combination with a CFTR amplifier compound with cells expressing a non-mutant or mutant CFTR in incubation media;
measuring CFTR levels and/or activity; and
identifying one or more compounds from the set of test compounds that modulate CFTR levels or activity, thereby identifying CFTR modulator compounds.

2. The method of claim 1, wherein the cells express non-mutant CFTR or at least one CFTR mutation selected from the group consisting of ΔF508, S549N, G542X, G551D, R117H, N1303K, W1282X, R553X, 621+1G>T, 1717-1G>A, 3849+10kbC>T, 2789+5G>A, 3120+1G>A, 1507del, R1162X, 1898+1G>A, 3659delC, G85E, D1152H, R560T, R347P, 2184insA, A455E, R334W, Q493X, Y122X, K710X, R553X, R709X, R1158X, R1162X and 2184delA.

3. The method of claim 1 or 2, wherein measuring comprises measuring CFTR activity.

4. The method of any one of claims 1-3, wherein the cells exogenously express a halide-sensitive yellow fluorescent protein (hsYFP).

5. The method of claim 4, wherein measuring CFTR activity comprises measuring the fluorescence of the hsYFP.

6. The method of claim 5, wherein measuring CFTR activity further comprises adding a halide salt to the cells, and detecting hsYFP signal quenching, thereby ascertaining the rate at which the halide salt is transported into the cells and CFTR activity.

7. The method of any one of claims 1-6, wherein measuring CFTR activity comprises measuring chloride transport activity using an electrophysiological assay.

8. The method of any one of claims 1-7, wherein incubating further comprises adding a CFTR potentiator compound to the cells.

9. The method of claim 8, wherein the CFTR potentiator is selected from the group consisting of ivacaftor, genistein, QBW251, GLPG2451, and GLPG1837.

10. The method of claim 1 or 2, wherein measuring comprises measuring CFTR levels within the cell or at the cell surface.

11. The method of claim 10, wherein the cells exogenously express a detectable CFTR fusion protein having at least one CFTR mutation selected from the group consisting of G542X, W1282X, Y122X, K710X, R553X, R709X, R1158X and R1162X.

12. The method of claim 10 or 11, wherein measuring CFTR levels comprises detecting the luminescence of the CFTR fusion protein using a luminescence assay.

13. The method of any one of claims 10-12, wherein the CFTR fusion protein is selected from the group consisting of CFTR-HRP and CFTR-luciferase.

14. The method of claim 10 or 11, wherein measuring CFTR levels comprises detecting the fluorescence of the CFTR fusion protein using a fluorescence assay.

15. The method of claim 14, wherein the CFTR fusion protein is selected from the group consisting of CFTR-RFP, CFTR-YFP and CFTR-GFP.

16. The method of claim 10, wherein the cells exogenously express a detectable, epitope-tagged CFTR protein having at least one CFTR mutation selected from the group consisting of G542X, W1282X, Y122X, K710X, R553X, R709X, R1158X and R1162X.

17. The method of claim 16, wherein measuring CFTR levels comprises detecting the epitope-tagged CFTR protein using an enzyme-linked immunosorbent assay.

18. The method of claim 16 or 17, wherein the epitope-tagged CFTR protein is selected from the group consisting of CFTR-HA and CFTR-FLAG.

19. The method of any one of claims 10-18, wherein the set of test compounds further includes a reference CFTR production corrector compound.

20. The method of claim 19, wherein the reference CFTR production corrector compound is G418 (geneticin).

21. The method of claim 1 or 2, wherein measuring CFTR levels comprises measuring mRNA levels of CFTR.

22. The method of any one of claims 1-21, wherein the CFTR modulator has a different mechanism of action than the CFTR amplifier.

23. The method of any one of claims 1-22, wherein the CFTR modulator is a CFTR potentiator or a CFTR corrector.
